(19) United States
(12) Patent Application Publication
(10) Pub. No.: US 2024/0390864 A1
(43) Pub. Date: Nov. 28, 2024

(54) MEMBRANE REACTOR AND METHOD OF OPERATING MEMBRANE REACTOR APPARATUS

(71) Applicant: NGK INSULATORS, LTD., Nagoya-City (JP)

(72) Inventors: Kenichi NODA, Nagoya-City (JP); Makiko ICHIKAWA, Nagoya-City (JP)

(73) Assignee: NGK INSULATORS, LTD., Nagoya-City (JP)

(21) Appl. No.: 18/792,716

(22) Filed: Aug. 2, 2024

Related U.S. Application Data
(63) Continuation of application No. PCT/JP2023/000765, filed on Jan. 13, 2023.

(30) Foreign Application Priority Data
Feb. 8, 2022 (JP) 2022-017640

(51) Int. Cl. B01D 69/14; B01D 53/22; B01D 71/02; C07C 1/12

(52) U.S. Cl. CPC B01D 69/145; B01D 53/228; B01D 71/0281; C07C 1/12; B01D 2256/245; B01D 2257/80; C07C 2529/76

(57) ABSTRACT

A membrane reactor includes a separation membrane complex and a catalyst. The separation membrane complex includes a separation membrane and a porous support. The catalyst accelerates a chemical reaction of a starting material. The support has a column-like shape extending in the longitudinal direction. The support has a membrane-formed cell (i.e., a first cell) having both longitudinal ends open. The first cell has an inner surface on which the separation membrane is arranged. The catalyst is arranged in the first cell of the separation membrane complex 1. The ratio of the average granule diameter of the catalyst to the inside diameter of the first cell is higher than or equal to 0.75 and lower than 1.

### CROSS-REFERENCE TO RELATED APPLICATIONS

The present application is a continuation application of International Application No. PCT/JP2023/000765 filed on Jan. 13, 2023, which claims the benefit of priority to Japanese Patent Application No. 2022-017640 filed on Feb. 8, 2022. The entire contents of these applications are incorporated herein by reference.

### TECHNICAL FIELD

The present invention relates to a membrane reactor and a method of operating membrane reactor apparatus including the membrane reactor.

### BACKGROUND ART

In order to reduce greenhouse gases, various technologies have been proposed in recent years for immobilizing carbon dioxide in a flue gas from a power generating plant or the like. As one example of such technologies, attention is placed on, for example, a technology for reacting carbon dioxide in a flue gas with hydrogen to produce methane (i.e., methanation). For example, methanation may be performed using membrane reactor apparatus that combines a separation membrane and a catalyst as disclosed in Japanese Patent Application Laid-Open No. 2018-008940 (Document 1).

In the membrane reactor apparatus, the separation membrane removes a reactant produced from a starting material by a chemical reaction occurring in the presence of the catalyst. This shifts reaction equilibrium of the chemical reaction toward the reactant side and accordingly improves the efficiency of producing the reactant. The membrane reactor apparatus is also used for purposes other than methanation, and membrane reactor apparatus having a variety of structures has been proposed (Japanese Patent Application Laid-Open No. 2019-156658 (Document 2) and Japanese Patent Application Laid-Open No. 2020-040030 (see Document 3).

Meanwhile, a monolith separation membrane complex is known as one example of a separation membrane for separating a specific substance from a plurality of types of substances. The separation membrane complex includes a monolith-type porous support including a plurality of cells, and a tube-like separation membrane formed on the inner surfaces of the cells. In the case of using this separation membrane complex in membrane reactor apparatus, it is conceivable to fill the cells (i.e., spaces on the inner side of the tube-like separation membrane) with catalysts. However, since the membrane reactor apparatus is ordinarily used at relatively high temperatures, stress may be caused by thermal expansion resulting from difference in thermal expansion coefficient or susceptibility to heating and cooling during temperature rise and drop between the catalyst and the separation membrane complex, and this may result in breakage of the separation membrane.

### SUMMARY OF THE INVENTION

The present invention is intended for a membrane reactor, and it is an object of the present invention to prevent breakage of a separation membrane resulting from difference in thermal expansion coefficient between a catalyst and a separation membrane complex.

A membrane reactor according to one preferable embodiment of the present invention includes a separation membrane complex including a separation membrane and a porous support, and a catalyst that accelerates a chemical reaction of a starting material. The support has a column-like shape extending in a longitudinal direction. The support includes a membrane-formed cell having at least one longitudinal end open and having an inner surface on which the separation membrane is formed. The catalyst is arranged in the membrane-formed cell of the separation membrane complex. A ratio of an average granule diameter of the catalyst to an inside diameter of the membrane-formed cell is higher than or equal to 0.75 and lower than 1.

This membrane reactor is capable of preventing breakage of the separation membrane resulting from difference in thermal expansion coefficient between the catalyst and the separation membrane complex.

Preferably, the ratio of the average granule diameter of the catalyst to the inside diameter of the membrane-formed cell is higher than or equal to 0.85 and lower than 1.

Preferably, the inside diameter of the membrane-formed cell is greater than or equal to 0.2 mm and less than or equal to 10 mm.

Preferably, the membrane-formed cell has a circular sectional shape perpendicular to the longitudinal direction.

Preferably, the membrane-formed cell has both longitudinal ends open.

Preferably, the separation membrane is a zeolite membrane.

Preferably, a zeolite constituting the zeolite membrane is composed of an 8- or less-membered ring at a maximum.

The present invention is also intended for a method of operating membrane reactor apparatus. In the method of operating membrane reactor apparatus according to one preferable embodiment of the present invention, the membrane reactor apparatus includes a membrane reactor, and a housing that includes the membrane reactor. The membrane reactor includes a separation membrane complex including a separation membrane and a porous support, and a catalyst that accelerates a chemical reaction of a starting material. The support has a column-like shape extending in a longitudinal direction. The support includes a membrane-formed cell having at least one longitudinal end open and having an inner surface on which the separation membrane is formed. The catalyst is arranged in the membrane-formed cell of the separation membrane complex. A ratio of an average granule diameter of the catalyst to an inside diameter of the membrane-formed cell is higher than or equal to 0.75 and lower than 1. The method of operating the membrane reactor includes a) supplying a starting material to the membrane-formed cell of the separation membrane complex; producing a reactant by chemically reacting the starting material in the presence of the catalyst in an environment with a temperature of 150° C. or higher; and separating a high-permeability substance in the reactant from the starting material by causing the high-permeability substance to permeate the separation membrane, and b) lowering a temperature of the membrane reactor to 40° C. or lower.

These and other objects, features, aspects and advantages of the present invention will become more apparent from the following detailed description of the present invention when taken in conjunction with the accompanying drawings.

### BRIEF DESCRIPTION OF DRAWINGS

FIG. 1 is a perspective view of a membrane reactor according to one embodiment.

FIG. 2 is a diagram showing one end face of the membrane reactor.

FIG. 3 is a perspective view of a separation membrane complex.

FIG. 4 is a diagram showing one end fac e of the separation membrane complex.

FIG. 5 is a sectional view of the separation membrane complex.

FIG. 6 is a sectional view of the membrane reactor.

FIG. 7 is a diagram showing another example of one end face of the separation membrane complex.

FIG. 8 is a flowchart showing the production of the membrane reactor.

FIG. 9 is a diagram showing membrane reactor apparatus.

FIG. 10 is a diagram showing a method of operating the membrane reactor apparatus.

### DETAILED DESCRIPTION

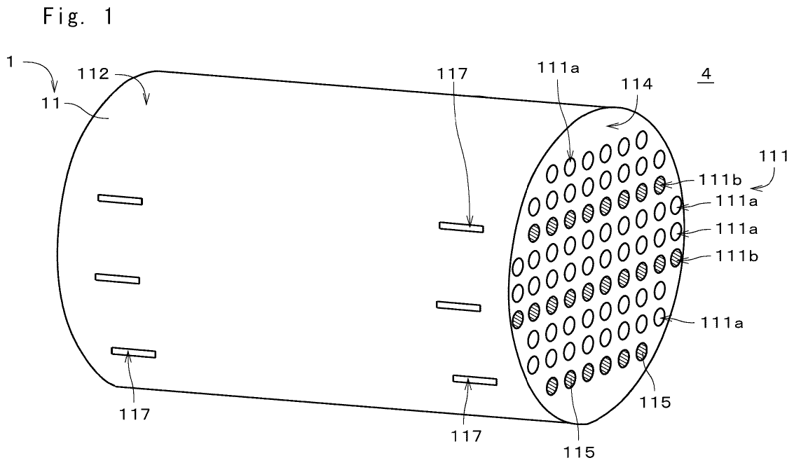

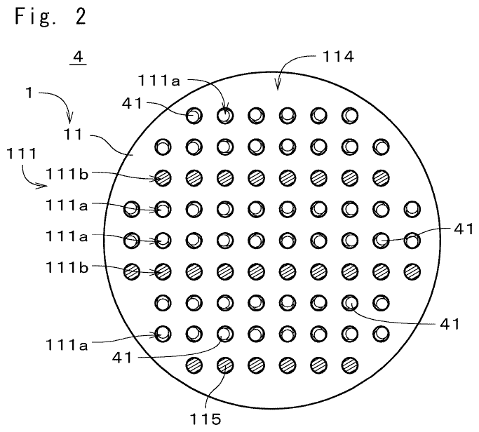

FIG. 1 is a perspective view of a membrane reactor 4 according to one embodiment of the present invention. FIG. 2 is a diagram showing one end face in the longitudinal direction of the membrane reactor 4 (i.e., approximately the right-left direction in FIG. 1). The membrane reactor 4 includes a separation membrane complex 1 and catalysts 41 kept in the separation membrane complex 1. The membrane reactor 4 produces a reactant by chemically reacting a starting material in the presence of the catalysts 41. The catalysts 41 are a substance that accelerates the chemical reaction of the starting material. The membrane reactor 4 separates a substance having high permeability through a separation membrane described later in the reactant from the starting material by causing the substance having high permeability to permeate the separation membrane. This further accelerates the chemical reaction of the starting material in the membrane reactor 4.

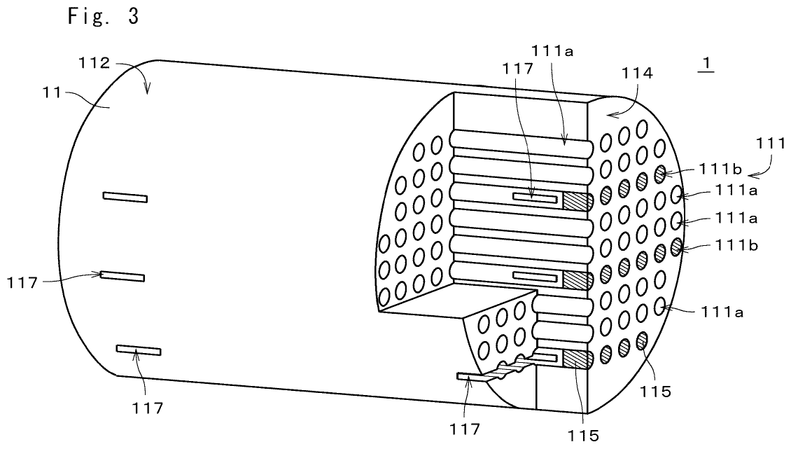

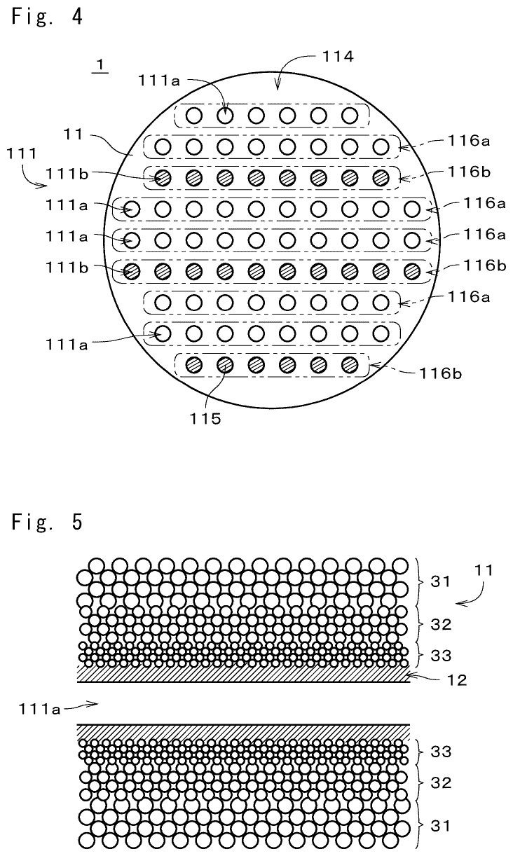

FIG. 3 is a perspective view of the separation membrane complex 1. FIG. 3 also shows part of the internal structure of the separation membrane complex 1. FIG. 4 is a diagram showing one end face 114 in the longitudinal direction of the separation membrane complex 1 (i.e., approximately the right-left direction in FIG. 3). FIG. 5 is a diagram showing part of a longitudinal section of the separation membrane complex 1 in enlarged dimensions and shows the vicinity of one cell 111, which will be described later. The separation membrane complex 1 separates a specific substance from a mixture of substances obtained by mixing a plurality of types of substances.

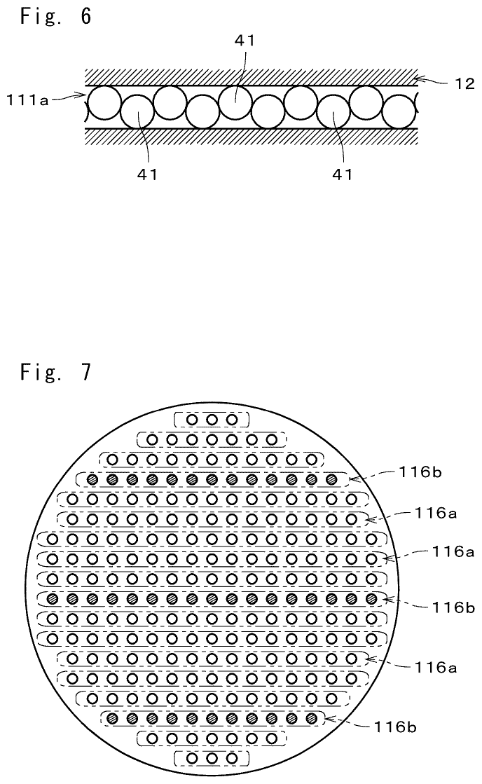

The separation membrane complex 1 includes a porous support 11 and a separation membrane 12 (see FIG. 5) formed on the support 11. In FIG. 5, the separation membrane 12 is cross-hatched (the same applies to FIG. 6 described later). The support 11 is a porous member that is permeable to gas and liquid. In the example shown in FIG. 3, the support 11 is a monolith support that includes an integrally-molded column-like body having a plurality of through holes 111 (hereinafter, also referred to as “cells 111”) each extending in the longitudinal direction of the body. In the support 11, the cells 111 are formed (i.e., partitioned) by a porous partition wall. In the example shown in FIG. 3, the support 11 has an approximately column-like outside shape. Each cell 111 may have, for example, an approximately circular cross-sectional shape perpendicular to the longitudinal direction. Note that “approximately circular” denotes a concept that includes not only a perfect circle but also an ellipse or a distorted circle. It is preferable that each cell 111 may have a perfect circular sectional shape, but this sectional shape does not necessarily need to be a perfect circle. The inside diameter of the cells 111 means the maximum diameter of a circle circumscribed around the sectional shape. In the illustration of FIG. 3, the diameter of the cells 111 is greater than the actual diameter, and the number of cells 111 is smaller than the actual number (the same applies to FIGS. 1, 2, and 4).

The cells 111 include first cells 111a and second cells 111b. In the example shown in FIGS. 1 to 4, the first cells 111a and the second cells 111b have approximately the same shape. The openings of the second cells 111b are plugged by a plugging member 115 in both longitudinal end faces 114 of the support 11. In other words, the second cells 111b have both longitudinal ends closed. In FIGS. 1 to 4, the plugging member 115 is cross-hatched. Meanwhile, the openings of the first cells 111a are not plugged but open in both of the longitudinal end faces 114 of the support 11.

The aforementioned separation membrane 12 (see FIG. 5) is formed on the inner surface of each first cell 111a having both longitudinal ends open. Preferably, the separation membrane 12 may be formed to cover the entire inner surface of each first cell 111a. That is, the first cells 111a are membrane-formed cells on the inner side of which the separation membrane 12 is formed. In the separation membrane complex 1, the separation membrane 12 is not formed on the inner side of the second cells 111b.

In the membrane reactor 4, as shown in FIG. 2, the catalysts 41 having an approximately spherical shape are arranged in the first cells 111a. Note that the catalysts 41 are not arranged in the second cells 111b. The catalysts 41 have a smaller granule diameter than the inside diameter of the first cells 111a as viewed in the longitudinal direction of the membrane reactor 4. In the membrane reactor 4, the first cells 111a are filled in a large number of granules of the catalysts 41. For example, the granules of the catalysts 41 may be formed by granulating fine powder of the catalysts 41. The shape and granule diameter of the catalysts 41 may be adjusted, for example, at the time of granulation or molding. As the catalysts 41, commonly known catalysts suitable for each reaction may be used and, for example, zirconia-supported nickel catalysts for methanation (i.e., catalysts with nickel (Ni) supported on stabilized zirconia) may be used. The type of the catalysts 41 is not limited to this example, and may be changed variously.

In the membrane reactor 4, one or both longitudinal end portions of the first cells 111a may be stuffed with a filling that does not plug the openings of the first cells 111a in order to prevent or inhibit coming off of the granules of the catalysts 41 from the inside of the first cells 111a. For example, the filling may be made of a soft material such as heat-resistant wool and partly blocks the openings of the first cells 111a while substantially not inhibiting the passage of gas and liquid. The filling becomes easily deformed by being pressed by the catalysts 41 when the catalysts 41 expand thermally as a result of a temperature rise in the membrane reactor 4.

The inside diameter of the first cells 111a may, for example, be greater than or equal to 0.2 mm and less than or equal to 10 mm. The inside diameter of the first cells 111a as used herein refers to the inside diameter of the first cells 111a that takes the thickness of the separation membrane 12 into consideration. In other words, the inside diameter of the approximately cylindrical separation membrane 12 formed on the inner surfaces of the first cells 111a may be greater than or equal to 0.2 mm and less than or equal to 10 mm.

The catalysts 41 may have any of various shapes. Examples of the shape of catalysts 41 include a spherical shape, an ellipsoidal shape, a cylinder-like shape (e.g., a circular cylinder-like shape, a prismatic shape, an oblique circular cylinder-like shape, or an oblique prismatic shape), and a conical shape (e.g., a circular conical shape or a pyramidal shape). It is preferable that the catalyst 41 may have a spherical shape, an ellipsoidal shape, or a cylinder-like shape because such a shape helps efficiently filling the inside of the first cells 111a without damaging the separation membrane 12. When the catalysts 41 have a spherical shape, the granule diameter of the catalysts 41 is the diameter of the sphere (or an average diameter of the sphere when the catalysts 41 have an approximately spherical shape). When the catalysts 41 have an ellipsoidal shape, the granule diameter of the catalysts 41 is the maximum diameter of a circle circumscribed around a section perpendicular to the major axis of the ellipsoid. When the catalysts 41 have a cylinder-like shape, the granule diameter of the catalyst 41 is the maximum diameter of a circle circumscribed around a section perpendicular to the axis parallel to the side face. When the catalysts 41 have a conical shape, the granule diameter of the catalysts 41 is the maximum diameter of a circle circumscribed around the bottom surface. The average granule diameter of the catalysts 41 is the median diameter (D₅₀) in the granule size distribution according to the volume standard. In the present embodiment, the catalysts 41 have an approximately spherical shape.

The ratio of the average granule diameter of the catalysts 41 to the inside diameter of the first cells 111a (hereinafter, also referred to as the “catalyst granules-size ratio”) may, for example, be higher than or equal to 0.75 and lower than 1 and preferably higher than or equal to 0.85 and lower than 1. If the catalyst granule-size ratio is set to be lower than 1, it is possible to easily fill the first cells 111a with the granules of the catalyst 41. If the catalyst granule-size ratio is set to be higher than or equal to 0.75, as shown in FIG. 6, a plurality of granules of the catalysts 41 are aligned one by one in the longitudinal direction (i.e., the right-left direction in FIG. 6) inside the first cells 111a, and it is possible to inhibit two or more granules of the catalysts 41 from being arranged at approximately the same longitudinal position. In other words, in the first cells 111a, two or more granules of the catalysts 41 are inhibited from being arranged side by side in a direction perpendicular to the longitudinal direction. FIG. 6 is a diagram showing part of a longitudinal section of the membrane reactor 4 in enlarged dimensions while omitting the illustration of the support 11. In the example shown in FIG. 6, each granule of the catalysts 41 comes in contact with the separation membrane 12 and longitudinally adjacent granules of the catalysts 41.

The granule size distribution of the catalysts 41 is measured after a large number of granules of the catalysts 41 are sifted through a sieve to remove fine fragments or the like. The aperture of the sieve is 1/20 of the aforementioned inside diameter of the first cells 111a. Accordingly, it is possible to inhibit fine fragments or the like of the catalysts 41 from affecting the calculation of the average granule diameter of the catalysts 41.

In the example shown in FIGS. 3 and 4, the cells 111 are arranged in the lengthwise direction (i.e., the up-down direction in FIG. 4) and the lateral direction in a matrix on the end faces 114 of the support 11. In the following description, a group of cells 111 that are arranged in a line in the lateral direction (i.e., the right-left direction in FIG. 4) is also referred to as a “cell line.” The cells 111 include a plurality of cell lines aligned in the lengthwise direction. In the example shown in FIG. 4, each cell line is composed of a plurality of first cells 111a or a plurality of second cells 111b.

In the example shown in FIG. 4, the cell lines are arranged such that one cell line of second cells 111b (hereinafter, also referred to as “second cell line 116b”) and two cell lines of first cells 111a (hereinafter, also referred to as “first cell lines 116a”) are alternately arranged adjacent to one another in the lengthwise direction. In FIG. 4, the first cell lines 116a and the second cell lines 116b are each enclosed by a chain double-dashed line (the same applies to FIG. 7, which will be described later). The second cell lines 116b plugged cell lines having both longitudinal ends plugged. A plurality of second cells 111b in each second cell line 116b communicate with one another via a slit 117 (see FIGS. 1 and 3) extending in the lateral direction. The slit 117 extends to the outer surface 112 of the support 11 on both lateral sides of the second cell line 116b, so that the second cells 111b in the second cell line 116b communicate with the space outside the support 11 via the slit 117. In other words, the slit 117 extends from the outer surface of the support 11 in the lateral direction through the second cell line 116b.

The first cell lines 116a are open cell lines having both longitudinal ends open and are also membrane-formed cell lines on the inner side of which the separation membrane 12 is formed (see FIG. 5). Two rows of first cells 111a that are adjacent to one lengthwise side of one second cell line 116b are an open cell line group. In other words, the open cell line group refers to two first cell lines 116 that are sandwiched between two second cell lines 116b that are located in closest proximity to each other in the lengthwise direction. The number of first cell lines 116a configuring one open cell line group is not limited to two, and may be changed variously. Preferably, the number of first cell lines 116a configuring one open cell line group may be greater than or equal to one and less than or equal to six and more preferably one or two. FIG. 7 shows an example in which five first cell lines 116a configure one open cell line group sandwiched between two second cell lines 116b.

The support 11 may have a longitudinal length of, for example, 100 mm to 2000 mm. The support 11 may have an outside diameter of, for example, 5 mm to 300 mm. The cell-to-cell distance between each pair of adjacent cells 111 (i.e., the thickness of the support 11 between portions of the adjacent cells 111 that are in closest proximity to each other) may be in the range of, for example, 0.3 mm to 10 mm. Surface roughness (Ra) of the inner surfaces of the first cells 111a of the support 11 may be in the range of, for example, 0.1 μm to 5.0 μm and preferably in the range of 0.2 μm to 2.0 μm.

Note that the shapes and dimensions of the support 11 and each cell 111 may be changed variously. For example, the cells 111 may have an approximately polygonal sectional shape perpendicular to the longitudinal direction. Even in this case, the inside diameter of the first cells 111a means the maximum diameter of a circle inscribed around. The first cells 111a and the second cells 111b may differ in shape and size. Moreover, some or all of the first cells 111a may differ in shape and size, and some or all of the second cells 111b may also differ in shape and size. In the case where some or all of the first cells 111a have different inside diameters, an arithmetical mean of the inside diameters of all of the first cells 111a is assumed to be the inside diameter of the first cells 111a.

The material for the support 11 may be any of various substances (e.g., ceramic or metal) as long as this substance has chemical stability in the process of forming the separation membrane 12 on the surface of the support 11. In the present embodiment, the support 11 is formed of a ceramic sintered body. Examples of the ceramic sintered body selected as the material for the support 11 include alumina, silica, mullite, zirconia, titania, yttria, silicon nitride and silicon carbide, and it is preferable that at least one of alumina, silica, and mullite may be included. In the present embodiment, the support 11 is formed of alumina.

The support 11 may contain an inorganic binder for binding aggregate particles of the aforementioned ceramic sintered body. As the inorganic binder, at least one of titania, mullite, easily sinterable alumina, silica, glass frit, clay minerals, and easily sinterable cordierite may be used.

The support 11 may have, for example, a multilayer structure in which a plurality of layers having different mean pore diameters are laminated one above another in the thickness direction in the vicinity of the inner surface of each first cell 111a as an open cell (i.e., in the vicinity of the separation membrane 12). In the example shown in FIG. 5, the support 11 includes a porous base material 31, a porous intermediate layer 32 formed on the base material 31, and a porous surface layer 33 formed on the intermediate layer 32. That is, the surface layer 33 is indirectly formed on the base material 31 via the intermediate layer 32. The intermediate layer 32 is formed between the base material 31 and the surface layer 33. The surface layer 33 configures the inner surface of each first cell 111a of the support 11, and the separation membrane 12 is formed on the surface layer 33. The surface layer 33 may have a thickness of, for example, 1 μm to 100 μm. The intermediate layer 32 may have a thickness of, for example, 100 μm to 500 μm. Note that the intermediate layer 32 and the surface layer 33 may or may not be formed on the inner surface of each second cell 111b. Also, the intermediate layer 32 and the surface layer 33 may or may not be formed on the outer surface 112 and the end faces 114 of the support 11.

The mean pore diameter of the surface layer 33 is smaller than the mean pore diameters of the intermediate layer 32 and the base material 31. The mean pore diameter of the intermediate layer 32 is smaller than the mean pore diameter of the base material 31. The mean pore diameter of the base material 31 may, for example, be greater than or equal to 1 μm and less than or equal to 70 μm. The mean pore diameter of the intermediate layer 32 may, for example be greater than or equal to 0.1 μm and less than or equal to 10 μm. The mean pore diameter of the surface layer 33 may, for example, be greater than or equal to 0.005 μm and less than or equal to 2 μm. The mean pore diameters of the base material 31, the intermediate layer 32, and the surface layer 33 can be measured by, for example, a mercury porosimeter, a perm porometer, or a nano-perm porometer.

The surface layer 33, the intermediate layer 32, and the base material 31 have approximately the same porosity. The porosities of the surface layer 33, the intermediate layer 32, and the base material 31 may, for example, be higher than or equal to 15% and lower than or equal to 70%. The porosities of the surface layer 33, the intermediate layer 32, and the base material 31 can be measured by, for example, the Archimedes method, mercury porosimetry, or image analysis.

The base material 31, the intermediate layer 32, and the surface layer 33 may be formed of the same material, or may be formed of different materials. For example, the base material 31 and the surface layer 33 may contain Al₂O₃ as a chief material. The intermediate layer 32 may contain aggregate particles that contain Al₂O₃ as a chief material, and an inorganic binder that contains TiO₂ as a chief material. In the present embodiment, the aggregate particles of the base material 31, the intermediate layer 32, and the surface layer 33 are substantially formed of only Al₂O₃. The base material 31 may contain an inorganic binder such as glass.

The average particle diameter of the aggregate particles in the surface layer 33 is smaller than the average particle diameter of the aggregate particles in the intermediate layer 32. The average particle diameter of the aggregate particles in the intermediate layer 32 is smaller than the average particle diameter of the aggregate particles in the base material 31. The average particle diameters of the aggregate particles in the base material 31, the intermediate layer 32, and the surface layer 33 can be measured by, for example, a laser diffraction method.

The plugging member 115 may be formed of a material similar to the material(s) for the base material 31, the intermediate layer 32, and the surface layer 33. The porosity of the plugging member 115 may be in the range of, for example, 15% to 70%.

As described above, the separation membrane 12 is formed on the inner surface of each first cell 111a as an open cell (i.e., on the surface layer 33) and covers approximately the entire inner surface. The separation membrane 12 is a porous membrane having microscopic pores. The separation membrane 12 separates a specific substance from a mixture of substances including a plurality of types of substances.

The separation membrane 12 may preferably be an inorganic membrane formed of an inorganic material, may more preferably be any of a zeolite membrane, a silica membrane, a carbon membrane, and a metal-organic framework (MOF) membrane, and may particularly preferably be a zeolite membrane. The zeolite membrane refers to at least a membrane obtained by forming a zeolite in membrane form on the surface of the support 11, and does not include a membrane obtained by just dispersing zeolite particles in an organic membrane. In the present embodiment, the separation membrane 12 is a zeolite membrane. The separation membrane 12 may be a zeolite membrane that contains two or more types of zeolites having different structures or compositions.

The separation membrane 12 may have a thickness of, for example, greater than or equal to 0.05 μm and less than or equal to 50 μm, preferably greater than or equal to 0.1 μm and less than or equal to 20 μm, and more preferably greater than or equal to 0.5 μm and less than or equal to 10 μm. Increasing the thickness of the separation membrane 12 improves separation performance. Reducing the thickness of the separation membrane 12 increases permeance. The surface roughness (Ra) of the separation membrane 12 may, for example, be less than or equal to 5 μm, preferably less than or equal to 2 μm, more preferably less than or equal to 1 μm, and yet more preferably less than or equal to 0.5 μm. The pore diameter of the separation membrane 12 may be in the range of, for example, 0.2 nm to 1 nm. The pore diameter of the separation membrane 12 is smaller than the mean pore diameter of the surface layer 33 of the support 11.

In the case where the zeolite constituting the separation membrane 12 is composed of an n-membered ring at the maximum, the minor axis of the n-membered ring pore is assumed to be the pore diameter of the separation membrane 12. In the case where the zeolite includes a plurality of types of n-membered ring pores where n is the same number, the minor axis of an n-membered ring pore having a largest minor axis is assumed to be the pore diameter of the separation membrane 12. Note that the n-membered ring refers to a ring in which n oxygen atoms compose the framework of each pore and each oxygen atom is bonded to T atoms described later to form a cyclic structure. The n-membered ring also refers to a ring that forms a through hole (channel), and does not include a ring that does not form a through hole. The n-membered ring pore refers to a pore formed of an n-membered ring. From the viewpoint of improving selectivity, it is preferable that the zeolite constituting the zeolite membrane 12 may be composed of an 8- or less-membered ring (e.g., 6- or 8-membered ring) at the maximum.

The pore diameter of the separation membrane 12 is uniquely determined by the framework structure of the zeolite and can be obtained from values disclosed in “Database of Zeolite Structures” [online], by International Zeolite Association, Internet <URL: http://www.iza-structure.org/databases/>.

There are no particular limitations on the type of the zeolite constituting the separation membrane 12, and the zeolite may, for example, be an AEI-, AEN-, AFN-, AFV-, AFX-, BEA-, CHA-, DDR-, ERI-, ETL-, FAU-(X-type, Y-type), GIS-, IHW-, LEV-, LTA-, LTJ-, MEL-, MFI-, MOR-, PAU-, RHO-, SOD-, or SAT-type zeolite. In the case where the zeolite is an 8-membered ring zeolite, the zeolite may, for example, be an AEI-, AFN-, AFV-, AFX-, CHA-, DDR-, ERI-, ETL-, GIS-, IHW-, LEV-, LTA-, LTJ-, RHO-, or SAT-type zeolite. In the present embodiment, the zeolite constituting the separation membrane 12 is a DDR-type zeolite.

The zeolite constituting the separation membrane 12 may contain, for example, at least one of silicon (Si), aluminum (Al), and phosphorus (P) as T atoms (i.e., atoms located in the center of oxygen tetrahedron (TO₄) that constitutes the zeolite). The zeolite constituting the separation membrane 12 may, for example, be a zeolite in which T atoms are composed of only Si or of Si and Al, an AlPO-type zeolite in which T atoms are composed of Al and P, an SAPO-type zeolite in which T atoms are composed of Si, Al, and P, an MAPSO-type zeolite in which T atoms are composed of magnesium (Mg), Si, Al, and P, or a ZnAPSO-type zeolite in which T atoms are composed of zinc (Zn), Si, Al, and P. Some of the T atoms may be replaced by other elements. The zeolite constituting the separation membrane 12 may contain alkali metal. The alkali metal may, for example, be sodium (Na) or potassium (K).

In the case where the zeolite constituting the separation membrane 12 contains Si atoms and Al atoms, the Si/Al ratio in the zeolite of the separation membrane 12 may, for example, be higher than or equal to one and lower than or equal to a hundred thousand. The Si/Al ratio refers to the molar ratio of Si elements to Al elements contained in the zeolite of the separation membrane 12. The Si/Al ratio may preferably be higher than or equal to 5, more preferably higher than or equal to 20, and yet more preferably higher than or equal to 100. It is preferable that the Si/Al ratio is as high as possible because the separation membrane 12 can achieve higher resistance to heat and acids. The Si/Al ratio can be adjusted by adjusting, for example, the compounding ratio of an Si source and an Al source in a starting material solution, which will be described later.

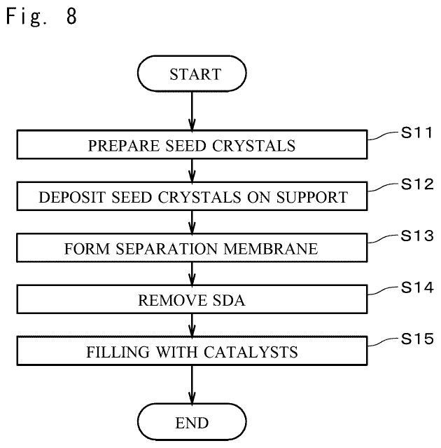

Next, one example of the procedure for the production of the membrane reactor 4 will be described with reference to FIG. 8. In the production of the membrane reactor 4, firstly, the separation membrane complex 1 is produced. In the production of the separation membrane complex 1, firstly, seed crystals used for forming the zeolite membrane 12 are synthesized and prepared (step S11). In the synthesis of the seed crystals, a starting material such as an Si source and a structure-directing agent (hereinafter, also referred to as an “SDA”) or the like are dissolved or dispersed in a solvent so as to prepare a starting material solution of the seed crystals. Then, the starting material solution is subjected to hydrothermal synthesis, and resultant crystals are washed and dried to obtain zeolite powder. The zeolite powder may be used as-is as the seed crystals, or may be subjected to processing such as pulverization to obtain the seed crystals.

Then, a dispersion obtained by dispersing the seed crystals in a solvent (e.g., water) is brought into contact with the inner surfaces of the first cells 111a of the support 11 so as to deposit the seed crystals in the dispersion on the inner surfaces of the first cells 111a (step S12). Note that the seed crystals may be deposited on the inner surfaces of the first cells 111a by any other technique. In step S12, for example, both of the longitudinal end portions of each second cell 111b may be plugged in advance.

Then, the support 11 with the seed crystals deposited thereon is immersed in a starting material solution. The starting material solution may be prepared by dissolving, for example, an Si source and an SDA in a solvent. As the solvent in the starting material solution, for example, water or alcohol such as ethanol may be used. The SDA contained in the starting material solution may, for example, be organic matter. As the SDA, for example, 1-adamantanamine may be used.

Then, the zeolite is grown by hydrothermal synthesis using the aforementioned seed crystals as nuclei, so that the zeolite membrane 12 is formed on the inner surface of each first cell 111a of the support 11 (step S13). Preferably, the temperature during hydrothermal synthesis may be in the range of 120° C. to 200° C. and may, for example, be 160° C. Preferably, the hydrothermal synthesis time may be in the range of 5 hours to 100 hours and may, for example, be 30 hours.

When the hydrothermal synthesis has ended, the support 11 and the zeolite membrane 12 are washed with deionized water. After washing, the support 11 and the zeolite membrane 12 may be dried at, for example, 80° C. After the drying of the support 11 and the zeolite membrane 12, the zeolite membrane 12 is subjected to heat treatment (i.e., firing) so as to almost completely remove the SDA in the zeolite membrane 12 by combustion and to perforate the zeolite membrane 12 with micropores. In this way, the aforementioned separation membrane complex 1 is obtained (step S14).

Thereafter, the inside of the first cells 111a of the separation membrane complex 1 is filled with the granules of the catalysts 41. Accordingly, the aforementioned membrane reactor 4 is obtained (step S15).

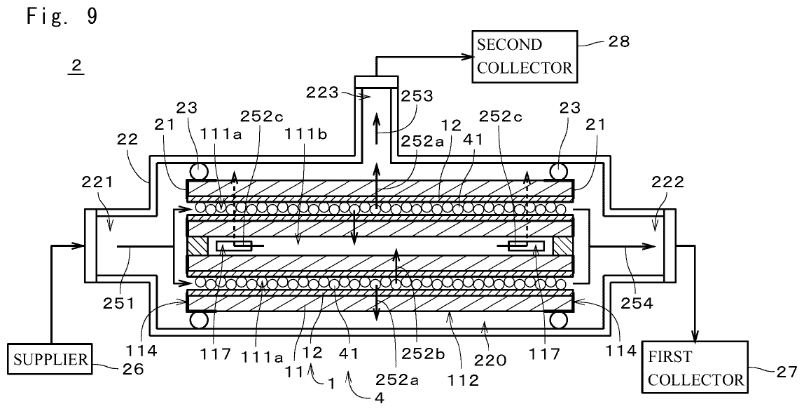

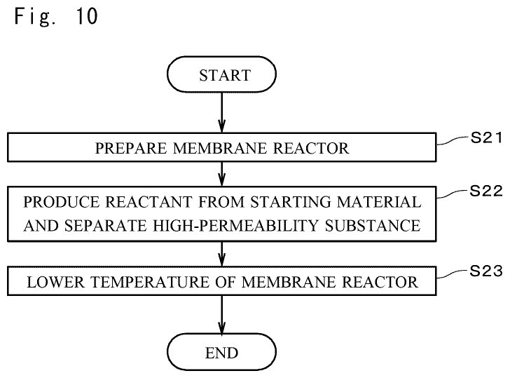

Next, a method of operating membrane reactor apparatus 2 including the aforementioned membrane reactor 4 will be described with reference to FIGS. 9 and 10. FIG. 9 is a sectional view showing the membrane reactor apparatus 2. To facilitate understanding of the drawing, FIG. 9 conceptually shows a section of the membrane reactor 4 in a simplified manner. FIG. 10 is a flowchart showing the operation of the membrane reactor apparatus 2.

In the membrane reactor apparatus 2, a reactant is produced by supplying a fluid as a starting material to the membrane reactor 4 and chemically reacting the starting material in the presence of the catalysts 41. Then, a substance having high permeability through the separation membrane 12 (hereinafter, also referred to as a “high-permeability substance”) in the reactant is separated from the starting material by being allowed to permeate the separation membrane complex 1. This accelerates the chemical reaction of the starting material in the membrane reactor 4. A substance having low permeability through the separation membrane 12 (hereinafter, also referred to as a “low-permeability substance”) in the reactant is less permeable through the separation membrane 12 and is thus difficult to be separated from the starting material. Note that the reactant does not necessarily need to include a low-permeability substance.

The starting material may be one type of gas or liquid, or a mixed gas containing a plurality of types of gases or a mixed solution containing a plurality of types of liquids, or a gas-liquid two-phase fluid that contains both gas and liquid.

The following description is given on the assumption that the starting material supplied to the membrane reactor apparatus 2 is a mixed gas that contains a plurality of types of gases, and the reactant produced from the starting material is also a mixed gas that contains a plurality of types of gases. For example, the starting material may be a mixed gas that contains carbon dioxide (CO₂) and hydrogen (H₂), and the membrane reactor apparatus 2 produces methane (CH₄) and water (H₂O) as reactants by chemical reaction. That is, methanation is performed in the membrane reactor apparatus 2. In the reactant, H₂O that is a high-permeability substance permeates the separation membrane 12, whereas CH₄ that is a low-permeability substance does not permeate the separation membrane 12. The catalysts 41 used for the above chemical reaction may, for example, be a zirconia-supported nickel catalyst.

The membrane reactor apparatus 2 includes the membrane reactor 4, a sealer 21, a housing 22, and two seal members 23. The membrane reactor 4, the sealer 21, and the seal member 23 are placed in the housing 22. In FIG. 9, the separation membrane 12 in the membrane reactor 4 is cross-hatched. The internal space of the housing 22 is an enclosed space isolated from the space around the housing 22. The housing 22 is connected to a supplier 26, a first collector 27, and a second collector 28.

The sealer 21 is a member that is formed on both ends in the longitudinal direction of the support 11 (i.e., the left-right direction in FIG. 9) and covers and seals both of the longitudinal end faces 114 of the support 11 and part of the outer surface 112 in the vicinity of both of the end faces 114. The sealer 21 prevents the inflow and outflow of gas and liquid from both of the end faces 114 of the support 11. For example, the sealer 21 may be a sealing layer made of glass or a resin. In the present embodiment, the sealer 21 is a glass seal having a thickness of 10 μm to 50 μm. The material and shape of the sealer 21 may be changed as appropriate. Note that the sealer 21 has a plurality of openings that overlap the plurality of first cells 111a of the support 11, so that both longitudinal ends of each first cell 111a are not covered with the sealer 21. This allows the inflow and outflow of fluid from both of the longitudinal ends into and out of the first cell 111a.

The housing 22 is an approximately cylindrical tube-like member. For example, the housing 22 may be made of stainless steel or carbon steel. The longitudinal direction of the housing 22 is approximately parallel to the longitudinal directions of the membrane reactor 4 and the separation membrane complex 1. One longitudinal end of the housing 22 (i.e., the end on the left side in FIG. 9) is provided with a supply port 221, and the other longitudinal end thereof is provided with a first exhaust port 222. The housing 22 further has a second exhaust port 223 on the side. The supply port 221 is connected to the supplier 26. The first exhaust port 222 is connected to the first collector 27. The second exhaust port 223 is connected to the second collector 28. Note that the shape and material of the housing 22 may be changed variously.

The two seal members 23 are placed along the entire circumference between the outer surface 112 of the separation membrane complex 1 and the inner surface of the housing 22 in the vicinity of both longitudinal ends of the membrane reactor 4. Each seal member 23 is an approximately circular ring-shaped member formed of a material that is impermeable to gas and liquid. For example, the seal members 23 may be O-rings or packing materials formed of a resin having flexibility. The seal members 23 are in tight contact with the outer surface 112 of the separation membrane complex 1 and the inner surface of the housing 22 along the entire circumference. In the example shown in FIG. 9, the seal members 23 are in tight contact with the outer surface of the sealer 21 between the end faces 114 of the support 11 and the slits 117 and are indirectly in tight contact with the outer surface 112 of the separation membrane complex 1 via the sealer 21. The space between each seal member 23 and the outer surface 112 of the separation membrane complex 1 and the space between each seal member 23 and the inner surface of the housing 22 are sealed so as to substantially disable the passage of gas and liquid. Note that the material for the seal members 23 may be carbon, metal, or any other inorganic material other than a resin.

The supplier 26 supplies a starting material, which is a mixed gas, into the internal space of the housing 22 via the supply port 221. For example, the supplier 26 may include a pressure mechanism such as a blower or a pump that sends the starting material toward the housing 22 under pressure. The pressure mechanism may include, for example, a temperature controller and a pressure regulator that respectively adjust the temperature and pressure of the starting material supplied to the housing 22. The first collector 27 and the second collector 28 may include, for example, a reservoir that stores the gas derived from the housing 22, or a blower or a pump that transfers the derived gas.

In the operation of the membrane reactor apparatus 2, firstly, the membrane reactor 4 is prepared (step S21). Specifically, the membrane reactor 4 is attached to the inside of the housing 22. Then, the supplier 26 supplies the starting material, which is a mixed gas, into the housing 22 (specifically, the space on the left side of the left end face 114 of the separation membrane complex 1) as indicated by an arrow 251. The starting material may contain, for example, CO₂ and H₂. The starting material may further contain a substance other than CO₂ and H₂. The pressure of the starting material supplied from the supplier 26 into the housing 22 (i.e., initial pressure) may be in the range of, for example, 0.1 MPa to 20 MPa. The temperature of the starting material supplied from the supplier 26 into the housing 22 may be in the range of, for example, 10° C. to 500° C. In the membrane reactor apparatus 2, the inside of the housing 22 is heated in advance, and the temperature of the membrane reactor 4 is raised to a temperature suitable for the chemical reaction of the starting material (e.g., a temperature of 150° C. to 500° C.). The membrane reactor 4 is maintained at this temperature during the chemical reaction of the starting material.

The starting material (e.g., CO₂ and H₂) supplied from the supplier 26 to the housing 22 flows into each first cell 111a of the separation membrane complex 1. In each first cell 111a, a reactant (e.g., CH₄ and H₂O) is produced by chemically reacting the starting material in the presence of the catalysts 41 in a high-temperature environment of 150° C. or higher. As indicated by arrows 252a, a high-permeability substance (e.g., H₂O) in the reactant permeates the separation membrane 12 and the support 11 from the first cells 111a and is derived to a separation space 220 around the separation membrane complex 1 from the outer surface 112 of the separation membrane complex 1. The separation space 220 is an approximately cylindrical space that is located radially outward of the outer surface 112 of the separation membrane complex 1 and sandwiched between the two seal members 23.

To be more specific, the high-permeability substance that has permeated the separation membrane 12 and the support 11 from the first cells 111a and flowed into the second cells 111b as indicated by arrows 252b is guided via the slits 117 to the outer surface 112 of the separation membrane complex 1 and derived to the separation space 220 as indicated by arrows 252c. The high-permeability substance that has flowed from the first cells 111a into the second cells 111b may permeate the support 11 and be derived to the separation space 220 without passing through the slits 117. Note that, since the end faces 114 of the support 11 are covered with the sealer 21, the separation membrane complex 1 prevents or inhibits the starting material from entering the inside of the support 11 through the end faces 114 and entering the separation space 220 without permeating the separation membrane 12.

As a result of the high-permeability substance (e.g., H₂O) permeating the separation membrane 12 and being derived to the separation space 220 as described above, the high-permeability substance is separated from other substances such as the starting material (e.g., CO₂ and H₂) and a low-permeability substance (e.g., CH₄) in the reactant (step S22). The substance derived from the outer surface 112 of the separation membrane complex 1 (hereinafter, also referred to as the “permeated substance”) is guided to and collected by the second collector 28 via the second exhaust port 223 as indicated by an arrow 253 in FIG. 9. The permeated substance may include a low-permeability substance or components of the starting material that have permeated the separation membrane 12, in addition to the aforementioned high-permeability substance.

Substances excluding the aforementioned permeated substance in the starting material and the reactant (hereinafter, also referred to as “non-permeated substances”) are guided to and collected by the first collector 27 via the first exhaust port 222 as indicated by an arrow 254. The non-permeated substances may include a high-permeability substance that has not permeated the separation membrane 12, in addition to the low-permeability substance and components of the starting material that have not been consumed by the aforementioned chemical reaction. For example, the non-permeated substances collected by the first collector 27 may be separated into the starting material and a low-permeability substance by a separation apparatus (not shown). The starting material obtained as a result of the separation by the separation apparatus may be circulated to the supplier 26 and supplied again into the housing 22. The low-permeability substance (e.g., CH₄) obtained as a result of the separation by the separation apparatus may be collected and used in various applications.

When the supply of the starting material has ended, the membrane reactor apparatus 2 stops the heating of the housing 22 and lowers the temperature of the membrane reactor 4. The temperature of the membrane reactor 4 drops to a temperature of 40° C. or lower (e.g., an ambient temperature) (step S23), and the operation of the membrane reactor apparatus 2 ends. Note that this temperature drop in the membrane reactor 4 in step S23 may be achieved by natural cooling or by forced cooling using, for example, air blowing or refrigerant.

In this way, in the operation of the membrane reactor apparatus 2, the temperature of the membrane reactor 4 rises and drops between a temperature of 40° C. or lower and a temperature of 150° C. or higher. During the temperature rise and drop in the membrane reactor 4, difference in thermal expansion coefficient between the separation membrane complex 1 and the catalysts 41 may cause stress occurring between the separation membrane complex 1 and the catalysts 41 and between the granules of the catalysts 41. For example, a case is assumed in which granules of two catalysts 41 having a granule diameter that is a half of the inside diameter of the first cells 111a are arranged side by side at approximately the same position in the longitudinal direction of the first cells 111a. In this case, if thermal expansion of the catalysts 41 is larger than thermal expansion of the separation membrane 12 during temperature rise, stress will be exerted between the granules of the two catalysts 41 in a direction approximately perpendicular to the longitudinal direction of the first cells 111a. Approximately similar stress will also be exerted when thermal shrinkage of the separation membrane 12 is larger than thermal shrinkage of the catalysts 41 during temperature drop. When such stress has occurred, a force directed radially outward (i.e., outward in the radial direction about a central axis extending in the longitudinal direction of the first cells 111a) may be applied to the separation membrane 12 and may press the separation membrane 12 against the support 11, and accordingly breakage may occur in the separation membrane 12.

In contrast, in the membrane reactor 4 according to the present invention, the catalyst granule-size ratio (i.e., the ratio of the average granule diameter of the catalysts 41 to the inside diameter of the first cells 111a) is set to be higher than or equal to 0.75 as described above. This allows the granules of the catalysts 41 to be aligned one by one in the longitudinal direction inside the first cells 111a and inhibits two or more granules of the catalysts 41 from being arranged at approximately the same longitudinal position. As a result, even if the thermal expansion of the catalysts 41 is larger than the thermal expansion of the separation membrane 12 during temperature rise, the stress between the granules of the catalyst 41 is directed in a direction along the longitudinal direction of the first cells 111a. Besides, since both of the longitudinal ends of the first cells 111a are open, the above stress will escape from those openings and substantially will not be directed in a direction perpendicular to the longitudinal direction. About the same can be said of the case where the thermal shrinkage of the separation membrane 12 is larger than the thermal shrinkage of the catalysts 41 during temperature drop. Accordingly, there is almost no radially outward force applied to the separation membrane 12, and this prevents breakage of the separation membrane 12 resulting from difference in thermal expansion coefficient between the catalysts 41 and the separation membrane complex 1.

Next, the relation between the characteristics of membrane reactors 4 in Examples 1 to 3 and the catalyst granule-size ratios in the membrane reactors 4 will be described with reference to Table 1. The same also applies to Comparative Example 1.

### Table 1

TABLE 1

| | Average Granule | Catalysts | |
|---|---|---|---|
| | Diameter of Catalysts | Granule- | Permeance |
| | (mm) | Size Ratio | Ratio |
| Example 1 | 1.9 | 0.95 | 1.0 |
| Example 2 | 1.7 | 0.85 | 1.1 |
| Example 3 | 1.5 | 0.75 | 2.1 |
| Comparative | 1.1 | 0.55 | 50.0 |
| Example 1 | | | |

In Example 1, the separation membrane complex 1 was prepared by a production method similar to the method including steps S11 to S14 described above. The support 11 was made of alumina and had an outside diameter of 30 mm, and the separation membrane 12 in each first cell 111a was a DDR-type zeolite membrane. The inside diameter of each first cell 111a was set to 2.0 mm. In step S15 described above, granules of approximately spherical zirconia-supported nickel catalysts were prepared as the catalysts 41, and each first cell 111a was filled with the catalysts 41 to obtain the membrane reactor 4. On the end faces 114 of the support 11, the sealer 21 made of glass was formed before deposition of seed crystals in step S12.

Then, the membrane reactor 4 was repeatedly caused a temperature rise and drop to simulate methanation using the membrane reactor apparatus 2. Specifically, five cycles of increasing the temperature of the membrane reactor 4 from ambient temperature (e.g., 25° C.) to 300° C. and decreasing the temperature from 300° C. to ambient temperature were performed by using a heater such as an electric furnace. The rate of temperature rise and the rate of temperature drop were both set to 100° C./h. Then, the performance of the separation membrane 12 in the membrane reactor 4 was evaluated before and after the five-cycle temperature rise/drop test.

In the performance evaluation of the separation membrane 12, the degree of defects (breakage) of the separation membrane 12 was evaluated. Thus, in the aforementioned membrane reactor apparatus 2, CF₄ was supplied into the housing 22 and permeance to CF₄ that permeated the separation membrane 12 in the membrane reactor 4 (hereinafter, also referred to as “CF₄ permeance”). Then, the ratio of CF₄ permeance after the temperature rise/drop test to CF₄ permeance before the temperature rise/drop test (hereinafter, also referred to as the “permeance ratio”) was obtained.

In the case where the permeance ratio was 1.0, it can be seen that there was no difference in the CF₄ permeability of the separation membrane 12 before and after the temperature rise/drop test, and as described above no breakage occurred in the separation membrane 12, resulting from difference in thermal expansion coefficient between the catalysts 41 and the separation membrane complex 1. On the other hand, in the case where the permeance ratio was higher than 1.0, there is the possibility that breakage may occur in the aforementioned separation membrane 12 and this may result in leakage of CF₄ from the breakage. This possibility will increase as the permeance ratio increases.

In Example 1, the average granule diameter of the catalysts 41 was 1.9 mm, and the catalyst granule-size ratio (i.e., the ratio of the average granule diameter of the catalysts 41 to the inside diameter of the first cells 111a) was 0.95. The permeance ratio was 1.0, and it can be thought that no breakage occurred in the separation membrane 12, resulting from difference in thermal expansion coefficient between the catalysts 41 and the separation membrane complex 1.

In Examples 2 and 3 and Comparative Example 1, the membrane reactor 4 was obtained in accordance with a procedure similar to that described in Example 1, except that the average granule diameter of the catalysts 41 was set to different values, and the performance of the separation membrane 12 was evaluated in accordance with a procedure similar to that described in Example 1.

In Example 2, the average granule diameter of the catalysts 41 was 1.7 mm, and the catalyst granule-size ratio was 0.85. The permeance ratio was 1.1, and it can be thought that almost no breakage occurred in the separation membrane 12, resulting from difference in thermal expansion coefficient between the catalysts 41 and the separation membrane complex 1.

In Example 3, the average granule diameter of the catalysts 41 was 1.5 mm, and the catalyst granule-size ratio was 0.75. The permeance ratio was 2.1, and it can be thought that breakage of the separation membrane 12 resulting from factors such as a difference in thermal expansion coefficient between the catalysts 41 and the separation membrane complex 1 was inhibited.

In Comparative Example 1, the average granule diameter the catalysts 41 was 1.1 mm, and the catalyst granule-size ratio was 0.55. The permeance ratio was 50.0 and high, and it can be thought that breakage occurred in the separation membrane 12, resulting from factors such as a difference in thermal expansion coefficient between the catalysts 41 and the separation membrane complex 1 and this resulted in leakage of CF₄ from the breakage.

Comparison of Examples 1 to 3 and Comparative Example 1 reveals that the catalyst granule-size ratio may preferably be higher than or equal to 0.75 from the viewpoint of inhibiting breakage of the separation membrane 12 resulting from difference in thermal expansion coefficient between the catalysts 41 and the separation membrane complex 1 (e.g., setting the permeance ratio to be lower than or equal to 10.0).

Comparison of Examples 1 to 3 reveals that the catalyst granule-size ratio may more preferably be higher than or equal to 0.85 from the viewpoint of further inhibiting breakage of the separation membrane 12 resulting from difference in thermal expansion coefficient between the catalysts 41 and the separation membrane complex 1 (e.g., setting the permeance ratio to be lower than or equal to 2.0).

As described above, the membrane reactor 4 includes the separation membrane complex 1 and the catalysts 41. The separation membrane complex 1 includes the separation membrane 12 and the porous support 11. The catalysts 41 accelerate chemical reactions of the starting material. The support 11 has a column-like shape extending in the longitudinal direction. The support 11 includes the membrane-formed cells (i.e., the first cells 111a) having both longitudinal ends open. The first cells 111a have inner surfaces on which the separation membrane 12 is formed. The catalysts 41 are arranged in the first cells 111a of the separation membrane complex 1. The ratio of the average granule diameter of the catalysts 41 to the inside diameter of the first cells 111a (i.e., the catalyst granule-size ratio) is higher than or equal to 0.75 and lower than 1. Accordingly, as described above, it is possible to inhibit breakage of the separation membrane 12 resulting from difference in thermal expansion coefficient between the catalysts 41 and the separation membrane complex 1.

Not only in the case where the first cells 111a have both longitudinal ends open, but also in the case where the first cells 111a have only one longitudinal end open, the membrane reactor 4 is capable of inhibiting breakage of the separation membrane 12 resulting from difference in thermal expansion coefficient between the catalyst 41 and the separation membrane complex 1 in approximately the same manner as described above. That is, if the first cells 111a have at least one longitudinal end open, the membrane reactor 4 is capable of inhibiting breakage of the aforementioned separation membrane 12. It is, however, noted that the first cells 111a may preferably have both longitudinal ends open from the viewpoint of further inhibiting breakage of the aforementioned separation membrane 12.

As described above, it is preferable that the ratio of the average granule diameter of the catalysts 41 to the inside diameter of the first cells 111a may be higher than or equal to 0.85 and lower than 1. In this case, the direction of stress that may possibly occur between the granules of the catalysts 4 during temperature rise and drop of the membrane reactor 4 during temperature rise or drop can become closer to a direction parallel to the longitudinal direction of the first cells 111a. Accordingly, it is possible to further inhibit breakage of the separation membrane 12 resulting from difference in thermal expansion coefficient between the catalysts 41 and the separation membrane complex 1.

As described above, it is preferable that the inside diameter of the first cells 111a may be greater than or equal to 0.2 mm and less than or equal to 10 mm. If the inside diameter of the first cells 111a is greater than or equal to 0.2 mm, it is possible to alleviate a shortage of the amount of the catalyst 41 retained in the first cells 111a. If the inside diameter of the first cells 111a is less than or equal to 10 mm, the high-permeability substance in the reactant produced in the radial central portions of the first cells 111a can more speedily arrive at the separation membrane 12 for separation. In other words, it is possible to efficiently remove the high-permeability substance from the inside of the first cells 111a. As a result, it is possible to further accelerate the chemical reaction of the starting material in the first cells 111a.

As described above, it is preferable that the first cells 111a may have a circular sectional shape perpendicular to the longitudinal direction. In this case, even if force is exerted on the separation membrane 12 from the catalysts 41 during temperature rise and drop in the membrane reactor 4, it is possible to disperse this force approximately evenly in the circumferential direction (i.e., the circumferential direction about a central axis extending in the longitudinal direction of the first cells 111a). In other words, it is possible to improve circumferential evenness of the thermal stress applied to the separation membrane 12. As a result, it is possible to further inhibit breakage of the separation membrane 12 resulting from difference in thermal expansion coefficient between the catalysts 41 and the separation membrane complex 1.

As described above, it is preferable that the separation membrane 12 may be a zeolite membrane. If the separation membrane 12 is composed of zeolite crystals having a uniform pore diameter, it is possible to favorably achieve selective permeation of a high-permeability substance. As a result, it is possible to efficiently separate a high-permeability substance from the starting material and a low-permeability substance and to remove the high-permeability substance from the inside of the first cells 111a. This further accelerates the chemical reaction of the starting material in the first cells 111a.

More preferably, the zeolite constituting the zeolite membrane may be composed of an 8- or less-membered ring at the maximum. In this case it is possible to more favorably achieve selective permeation of a high-permeability substance such as H₂O that has a relatively small molecular size. As a result, it is possible to further accelerate the chemical reaction of the starting material in the first cells 111a.

The membrane reactor apparatus 2 described above includes the membrane reactor 4 and the housing 22 that includes the membrane reactor 4. As described above, the membrane reactor 4 includes the separation membrane complex 1 and the catalysts 41. The separation membrane complex 1 includes the separation membrane 12 and the porous support 11. The catalysts 41 accelerate chemical reactions of the starting material. The support 11 has a column-like shape extending in the longitudinal direction. The support 11 includes the membrane-formed cells (i.e., the first cells 111a) having both longitudinal ends open. The first cells 111a have an inner surface on which the separation membrane 12 is formed. The catalysts 41 are arranged in the first cells 111a of the separation membrane complex 1. The ratio of the average granule diameter of the catalysts 41 to the inside diameter of the first cells 111a (i.e., the catalyst granule-size ratio) is higher than or equal to 0.75 and lower than 1. The method of operating the membrane reactor apparatus 2 includes the step of supplying a starting material to the first cells 111a of the separation membrane complex 1, producing a reactant by chemically reacting the starting material 1 in the presence of the catalysts 41 in an environment at a temperature of 150° C. or higher, and separating a high-permeability substance in the reactant from the starting material by allowing the high-permeability substance to permeate the separation membrane 12 (step S22), and the step of lowering the temperature of the membrane reactor 4 to a temperature of 40° C. or less (step S23). As described above, the membrane reactor 4 is capable of inhibiting breakage of the separation membrane 12 resulting from difference in thermal expansion coefficient between the catalysts 41 and the separation membrane complex 1. Therefore, the structure of the membrane reactor 4 is particularly suitable for use in the membrane reactor apparatus 2 described above.

The membrane reactor 4 and the method of operating the membrane reactor apparatus 2 described above may be modified in various ways.

For example, the inside diameter of the first cells 111a may be less than 0.2 mm, or may be greater than 10 mm.

In the separation membrane complex 1, the zeolite constituting the separation membrane 12, which is a zeolite membrane, may be composed of a more than 8-membered ring at the maximum. The separation membrane 12 is not limited to a zeolite membrane, and may be an inorganic membrane such as a silica membrane or a carbon membrane, or may be an organic membrane such as a polyimide membrane or a silicone membrane. The separation membrane complex 1 may further include a function membrane or a protection membrane that is laminated on the separation membrane 12, in addition to the separation membrane 12. Such a function membrane or a protection membrane may be a zeolite membrane, may be an inorganic membrane other than a zeolite membrane, or may be an organic membrane.

The structure of the separation membrane complex 1 is not limited to the examples described above and may be modified in various ways. For example, the slits 117 that penetrate the second cells 111b may be omitted. The cells 111 placed in the support 11 do not necessarily include the second cells 111b having both longitudinal ends plugged, and all of the cells 111 may have both ends open and have an inner surface on which the separation membrane 12 is formed. In other words, all of the cells 111 may be the first cells 111a. The number of first cells 111a may be one.

The membrane reactor apparatus 2 may cause a chemical reaction other than methanation. For example, the chemical reaction may be a reverse shift reaction, a methanol synthesis reaction, or a Fischer-Tropsch synthesis reaction.

The aforementioned method of operating the membrane reactor apparatus 2 may be applied to the operation of membrane reactor apparatus that differs in structure from the aforementioned membrane reactor apparatus 2. The membrane reactor 4 may be used in the membrane reactor apparatus 2 that is operated by a method other than the aforementioned operation method. The membrane reactor 4 may also be used in membrane reactor apparatus that differs in structure from the aforementioned membrane reactor apparatus 2.

The configurations of the above-described preferred embodiment and variations may be appropriately combined as long as there are no mutual inconsistencies.

While the invention has been shown and described in detail, the foregoing description is in all aspects illustrative and not restrictive. It is therefore understood that numerous modifications and variations can be devised without departing from the scope of the invention.

### INDUSTRIAL APPLICABILITY

The membrane reactor according to the present invention is usable in, for example, membrane reactor apparatus that produces various reactants from various starting materials by chemical reactions caused in the presence of catalysts.

### REFERENCE SIGNS LIST

1 separation membrane complex
2 membrane reactor apparatus
4 membrane reactor
11 support
12 separation membrane
22 housing
41 catalyst
111a first cell
S11 to S15, S21 to S23 step

1. A membrane reactor comprising:
a separation membrane complex including a separation membrane and a porous support; and
a catalyst that accelerates a chemical reaction of a starting material,
wherein said support has a column-like shape extending in a longitudinal direction,
said support includes a membrane-formed cell having at least one longitudinal end open and having an inner surface on which said separation membrane is formed,
said catalyst is arranged in said membrane-formed cell of said separation membrane complex, and
a ratio of an average granule diameter of said catalyst to an inside diameter of said membrane-formed cell is higher than or equal to 0.75 and lower than 1.

2. The membrane reactor according to claim 1, wherein
the ratio of the average granule diameter of said catalyst to the inside diameter of said membrane-formed cell is higher than or equal to 0.85 and lower than 1.

3. The membrane reactor according to claim 1, wherein
the inside diameter of said membrane-formed cell is greater than or equal to 0.2 mm and less than or equal to 10 mm.

4. The membrane reactor according to claim 1, wherein
said membrane-formed cell has a circular sectional shape perpendicular to the longitudinal direction.

5. The membrane reactor according to claim 1, wherein
said membrane-formed cell has both longitudinal ends open.

6. The membrane reactor according to claim 1, wherein
said separation membrane is a zeolite membrane.

7. The membrane reactor according to claim 6, wherein
a zeolite constituting said zeolite membrane is composed of an 8- or less-membered ring at a maximum.

8. A method of operating membrane reactor apparatus,
said membrane reactor apparatus including:
a membrane reactor; and
a housing that includes said membrane reactor,
said membrane reactor including:
a separation membrane complex including a separation membrane and a porous support; and
a catalyst that accelerates a chemical reaction of a starting material,
wherein said support has a column-like shape extending in a longitudinal direction,
said support includes a membrane-formed cell having at least one longitudinal end open and having an inner surface on which said separation membrane is formed,
said catalyst is arranged in said membrane-formed cell of said separation membrane complex,
a ratio of an average granule diameter of said catalyst to an inside diameter of said membrane-formed cell is higher than or equal to 0.75 and lower than 1,
the method of operating said membrane reactor equipment comprising:
a) supplying a starting material to said membrane-formed cell of said separation membrane complex; producing a reactant by chemically reacting said starting material in the presence of said catalyst in an environment with a temperature of 150° C. or higher; and separating a high-permeability substance in said reactant from said starting material by causing said high-permeability substance to permeate said separation membrane; and
b) lowering a temperature of said membrane reactor to 40° C. or lower.
